(19) United States
(12) Patent Application Publication
(10) Pub. No.: US 2026/0091075 A1
(43) Pub. Date: Apr. 2, 2026

(54) METHOD FOR IMPROVING SKIN CONDITION AND/OR RELIEVING ANEMIA BY PEACH EXTRACT

(71) Applicant: TCI CO., LTD., Taipei (TW)

(72) Inventors: YUNG-HSIANG LIN, Taipei (TW); FAN-JHEN DAI, Taipei (TW); YIN-DI SU, Taipei (TW)

(21) Appl. No.: 18/963,781

(22) Filed: Nov. 29, 2024

(30) Foreign Application Priority Data
Sep. 30, 2024 (TW) 113137442

(51) Int. Cl. A61K 36/736; A61K 8/60; A61K 8/9789; A61K 31/7048; A61P 7/06; A61P 17/18; A61Q 19/08

(52) U.S. Cl. CPC A61K 36/736; A61K 8/602; A61K 8/9789; A61K 31/7048; A61P 7/06; A61P 17/18; A61Q 19/08

(57) ABSTRACT

A method for improving skin condition and/or relieving anemia of a subject in need thereof includes administering to the subject an effective amount of peach extract, where the peach extract is extracted from red flesh of peaches. The peach extract has at least one of the following effects of: resisting blue light-induced injury, improving oxidation resistance of skin fibrocytes, improving activity of mitochondria, promoting production of skin elastin, improving skin texture, reducing skin wrinkles, minimizing skin trichopores, improving the content of heme, and improving the content of iron in blood.

### CROSS-REFERENCE TO RELATED APPLICATION

This non-provisional application claims priority under 35 U.S.C. § 119 (a) to patent application No. 113137442 filed in Taiwan, R.O.C. on Sep. 30, 2024, the entire contents of which are hereby incorporated by reference.

### BACKGROUND

### Technical Field

The present invention relates to a method of using a peach extract, and in particular, to a method for improving skin condition and/or relieving anemia of a subject in need by the peach extract thereof.

### Related Art

Peaches (Prumuis persica), also known as blood peaches, belong to P. subg. Amygdalus, Prunus, Rosaceae.

In addition to health, modern women are pursuing more delicate skin and ruddy complexion. Therefore, more diverse and natural raw materials are developed to assist women in pursuing beauty.

### SUMMARY

In view of this, the present invention provides use of a peach extract in the preparation of a composition for improving skin condition and/or relieving anemia. The present invention provides a method for improving skin condition of a subject in need thereof, including administering to the subject an effective amount of peach extract, where the peach extract is extracted from red flesh of peaches. The present invention also provides a method for relieving anemia of a subject in need thereof, including administering to the subject an effective amount of the peach extract, where the peach extract is extracted from red flesh of peaches.

In some embodiments, provided is method for improving skin condition of a subject in need, comprising administering to the subject an effective amount of a peach extract, where the peach extract is extracted from red flesh of peaches.

In some embodiments, the peach extract resists blue light-induced injury of the subject to achieve the effect of improving skin condition.

In some embodiments, the peach extract improves activity of mitochondria of the subject to achieve the effect of improving skin condition.

In some embodiments, the peach extract promotes production of skin elastin of the subject to achieve the effect of improving skin condition.

In some embodiments, the peach extract improves skin texture of the subject to achieve the effect of improving skin condition.

In some embodiments, the peach extract reduces skin wrinkles of the subject to achieve the effect of improving skin condition.

In some embodiments, the peach extract minimizes skin trichopores of the subject to achieve the effect of improving skin condition.

In some embodiments, the peach extract improves oxidation resistance of skin fibrocytes of the subject to achieve the effect of improving skin condition.

In some other embodiments, provided is method for relieving anemia of a subject in need, comprising administering to the subject an effective amount of a peach extract, where the peach extract is extracted from red flesh of peaches.

In some embodiments, the peach extract improves the content of heme of the subject to achieve the effect of relieving anemia.

In some embodiments, the peach extract improves the content of iron in blood of the subject to achieve the effect of relieving anemia.

In some embodiments, the peach extract includes at least hyperoside, isoquercitrin, nicotiflorin, and rutin.

In some embodiments, the hyperoside, the isoquercitrin, the nicotiflorin, and the rutin resist blue light-induced injury of the subject to achieve the effect of relieving anemia.

In some embodiments, the hyperoside, the isoquercitrin, the nicotiflorin, and the rutin improve oxidation resistance of skin fibrocytes of the subject to achieve the effect of relieving anemia.

In conclusion, according to any embodiment of the present invention, the peach extract can be used in the preparation of a composition for improving skin condition or relieving anemia. In some embodiments, the peach extract further has at least one of the following effects: resisting blue light-induced injury, improving oxidation resistance of skin fibrocytes, improving activity of mitochondria, promoting production of skin elastin, improving skin texture, reducing skin wrinkles, minimizing skin trichopores, improving the content of heme, improving the content of iron in blood, and the like.

### BRIEF DESCRIPTION OF THE DRAWINGS

FIG. 1 is a bar chart showing the results of a blue light resistance test of a peach extract;

FIG. 2 is a bar chart showing the results of an antioxidant activity test of the peach extract;

FIG. 3 is a bar chart showing the results of a test for relative content of elastin of the peach extract;

FIG. 4 is a bar chart showing the results of a test for relative activity of mitochondria of the peach extract;

FIG. 5 is a bar chart showing the results of a test for human body skin wrinkle amount of the peach extract;

FIG. 6 is a bar chart showing the results of a test for human body skin texture amount of the peach extract;

FIG. 7 is a bar chart showing the results of a test for human body relative amount of trichopores of the peach extract;

FIG. 8 is a bar chart showing the results of a test for the content of heme in human body blood of the peach extract;

FIG. 9 is a bar chart showing the results of a test for the content of iron in human body blood of the peach extract;

FIG. 10 is a dendrogram showing the analysis results of bioactive substances in the peach extract;

FIG. 11 is an HPLC spectrum of the peach extract;

FIG. 12 is a bar chart showing the results of a blue light resistance test of the bioactive substances in the peach extract; and

FIG. 13 is a bar chart showing the results of an antioxidant activity test of the bioactive substances in the peach extract.

### DETAILED DESCRIPTION

As used herein, the concentration symbol “wt %” generally refers to the concentration in percentage by weight, while the concentration symbol “vol %” generally refers to the concentration in percentage by volume. As used herein, the term “extract” refers to the product prepared by extraction. The extract may be dissolved in a solvent to be presented as a solution, or the extract may be presented as a concentrate or essence free or substantially free of solvent. As used herein, the term “subject” refers to an organism in need thereof, such as human beings.

In some embodiments, a peach extract is obtained by carrying out extraction on peaches with a solvent or by at least one post-treatment process including filtration, concentration, sterilization, and the like, after extraction, where the peaches are ripe fruits of a peach plant (scientific name: Prunus persica). The extraction solvent may be water, alcohols, or a combination thereof.

In some embodiments, the peach extract is prepared by carrying out extraction on the peaches with water as an extraction solvent to obtain an initial extract, filtering the initial extract to remove solid particles to obtain a filtrate (hereinafter referred to as a first filtrate), concentrating the first filtrate to obtain a concentrated product (which may be a solid and/or a liquid), and sterilizing the concentrated product to obtain a sterile product.

In some embodiments, the peaches have red flesh having the Pantone color scale of 504C to 506C. In some embodiments, the peaches are fresh or frozen. In some embodiments, the peaches may refer to an entire peach (that is, a fruit having a pericarp, a flesh, and a pit), peach flesh with a pericarp from an entire pitted peach, or peach flesh without a pericarp from an entire pitted and peeled peach. For example, the peaches may be flesh with pericarps from frozen peaches imported from France (which do not have pits).

In some embodiments, the peaches may be pieces or puree of peaches that have been subjected to at least one physical processing procedure of cutting, pulverizing, grinding, pressing, and the like to change their appearance. For example, the peaches are pieces of peaches obtained by pulverizing flesh with a pericarp from frozen peaches, and the pieces may have the diameter less than or equal to 12 mm. The peach pieces may be obtained by pulverizing peaches into coarse pieces with a pulverizer and sieving the coarse pieces with a sieve having the pore size of about 12 mm.

In some embodiments, the initial extract is obtained by carrying out extraction on the peaches with water at a ratio of 1:(3-8). In some embodiments, the initial extract is obtained by carrying out extraction on the peaches with water at a ratio of 1:5.

In some embodiments, the extraction refers to heating a mixture of the peaches and the water to a specific temperature, holding for a specific period of time. In some embodiments, the extraction refers to heating water to a specific temperature, adding peaches, and then mixing the peaches with the water at the specific temperature, holding for a specific period of time. In an embodiment, the specific temperature is between 100° C. and 75° C. In an embodiment, the specific temperature is 85±5° C. In an embodiment, the specific period of time is 40 minutes to 80 minutes. In an embodiment, the specific period of time is 60 minutes. For example, water is heated to 85±5° C. and then pieces of flesh with pericarps from frozen peaches imported from France are added, mixed, and held at the temperature of 85±5° C. for 60 minutes to obtain a peach extract.

In an embodiment, the extraction further includes an acceptance procedure, and the acceptance procedure is performed by measuring the degree Brix of the initial extract and completing the acceptance procedure when the degree Brix of the initial extract is measured to be greater than or equal to 1.2 Brix°. In an embodiment, the acceptance procedure further includes extending specific time when the degree Brix of the initial extract is measured to be less than 1.2 Brix°, measuring the degree Brix of the initial extract again after extending the specific time, and completing the acceptance procedure by repeatedly extending time and measuring the degree Brix until the degree Brix of the initial extract is measured to be greater than or equal to 1.2 Brix°.

In some embodiments, when the concentrated product is a liquid, the concentrated product may be first filtered one or more times to obtain a filtrate (hereinafter referred to as a second filtrate), and the second filtrate may be sterilized to obtain a sterile product. It should be understood that the “peach extract” herein may be any one of the initial extract, the filtrate, the concentrated product, and the sterile product obtained in the preparation process.

For example, red flesh with pericarps from pitted frozen peaches imported from France is pulverized into small peach particles, then the peach particles and water are mixed at 85±5° C. at a weight ratio of the peach particles to the water of 1:5, and then extraction is performed at 85±5° C. for 0.5-2 hours to obtain an initial extract. Then the initial extract is filtered through a 400-mesh sieve to obtain a filtrate, and then the filtrate is concentrated under reduced pressure at 60±5° C. and a pressure of 1±0.2 kg/cm² to obtain a concentrated product. Then the concentrated product is filtered and sterilized to obtain a peach extract.

In some embodiments, the degree Brix of the initial extract is greater than or equal to 1.2 Brix°. In some embodiments, the degree Brix of the concentrated product is about 9±0.5 Brix°. In some embodiments, the pH value of the peach extract is less than or equal to 3.7. In some embodiments, the acidity of the peach extract is 1.3±0.5. In some embodiments, the peach extract includes malic acid, and the acidity is adjusted to 1.3±0.5 by using the malic acid.

In some embodiments, the peach extract can be used for improving skin condition, and the peach extract is extracted from red flesh (with or without a pericarp) of peaches.

In an embodiment, the peach extract can be used to resist blue light-induced injury. In an embodiment, the peach extract can significantly reduce blue light-induced injury of skin fibrocytes at a concentration of 0.125 mg/mL or at a concentration of 0.0625 mg/mL.

In an embodiment, the peach extract can be used to improve oxidation resistance of skin fibrocytes. In an embodiment, compared with no treatment with the peach extract, the peach extract can significantly improve oxidation resistance of skin fibrocytes at a concentration of 0.125 mg/mL or at a concentration of 0.0625 mg/mL.

In an embodiment, the peach extract can be used to improve activity of mitochondria. In an embodiment, compared with no treatment with the peach extract, the peach extract can significantly improve activity of mitochondria in skin fibrocytes at a concentration of 0.125 mg/mL or at a concentration of 0.0625 mg/mL.

In an embodiment, the peach extract can be used to promote production of skin elastin. In an embodiment, compared with no treatment with the peach extract, the peach extract can significantly promote production of skin elastin in skin fibrocytes at a concentration of 0.125 mg/mL or at a concentration of 0.0625 mg/mL.

In an embodiment, the peach extract can be used to improve skin texture. In an embodiment, the peach extract can be used to reduce skin wrinkles. In an embodiment, the peach extract can be used to minimize skin trichopores. In an embodiment, the peach extract can be used to improve the content of heme. In an embodiment, the peach extract can be used to improve the content of iron in blood. In an embodiment, an effective amount of the peach extract used is 0.9 g/day.

In some embodiments, any one of the compositions may be a pharmaceutical product. In other words, the pharmaceutical product contains an effective amount of the peach extract.

In some embodiments, the pharmaceutical product may be prepared by techniques well known to a person skilled in the art into a dosage form suitable for enteral, parenteral, oral, or topical administration.

In some embodiments, the dosage form for enteral or oral administration may be, but is not limited to, a tablet, a troche, a lozenge, a pill, a capsule, dispersible powder or granules, a solution, a suspension, an emulsion, a syrup, an elixir, a slurry, or the like. In some embodiments, the dosage form for parenteral or topical administration may be, but is not limited to, an injection, sterile powder, an external preparation, or the like. In some embodiments, the administration mode of the injection may be, but is not limited to, a subcutaneous injection, an intraperitoneal injection, an intradermal injection, or an intralesional injection.

In some embodiments, the pharmaceutical product may contain a pharmaceutically acceptable carrier widely used in the pharmaceutical manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome, and the like. The type and amount of the carrier selected for use are within the specialty literacy and routine skill of a person skilled in the art. In some embodiments, the solvent as a pharmaceutically acceptable carrier may be water, normal saline, phosphate buffered saline (PBS), or an aqueous solution containing alcohol.

In some embodiments, any one of the compositions may be an edible composition. In other words, the edible composition contains a specific content of the peach extract. In some embodiments, the edible composition may be a food product or a food additive. In some embodiments, the food product may be, but not limited to, beverages, fermented foods, bakery products, health foods, and dietary supplements.

In some embodiments, any one of the compositions may be a cosmetic or a skin-care product. In other words, the cosmetic or the skin-care product contains a specific content of the peach extract.

In some embodiments, the cosmetic or the skin-care product may be in any of the following forms: a lotion, a gel, a jelly mask, a mudpack, an emulsion, a cream, a lipstick, a foundation, a pressed powder, a loose powder, a cleansing oil, a makeup removing lotion, a facial cleanser, a body shampoo, a shampoo, a hair conditioner, a sunscreen lotion, a hand cream, a nail polish, a perfume, an essence and a facial mask. In some embodiments, the cosmetic or the skin-care product may further contain an acceptable ingredient for external products as necessary. In some embodiments, the acceptable ingredient for external products may be, for example, an emulsifier, a penetration enhancer, a softener, a solvent, an excipient, an antioxidant, or a combination thereof.

In all the following tests, a student t-test is used to determine whether there is a statistically significant difference between two sample groups, that is, their p values are calculated.

Unless otherwise indicated, the following tests are all performed at room temperature. The room temperature herein is 25±5° C.

### Example I Preparation of Peach Extract

### Peaches:

Peaches with red flesh of the color scale of 504C to 506C were selected from frozen peaches imported from France, and the selected peaches were pitted and coarsely pulverized with the pore size of 12 mm to obtain pulverized peach flesh with a pericarp.

### Preparation Process:

First, water (RO water) at 5 times the weight of the peaches was heated, when the water temperature reached 85° C., the peaches were added, then extraction was performed while the water temperature was continuously maintained at 85±5° C. to obtain an initial extract and the degree Brix of the initial extract was measured. When the degree Brix of the initial extract was measured to be greater than 1.2 Brix°, the subsequent steps were performed. Herein, the extraction time was 60 minutes.

Then the initial extract was filtered through a 400-mesh sieve to obtain a filtrate, and then the filtrate was concentrated under reduced pressure at 60±5° C. and a pressure of 1±0.2 kg/cm² to obtain a concentrated product. Herein, the degree Brix of the concentrated product was 9 Brix°.

Then, the concentrated product was filtered by using a 400-mesh sieve and a filter with the pore size of 5 μm to obtain the peach extract.

### Example II Blue Light-Induced Injury Resistance Test

### Materials and Instruments:

1. Cell line: human skin fibroblasts CCD-966sk, obtained from the Bioresource Collection and Research Center (BCRC); Cat. 60153, hereinafter referred to as human skin fibrocytes.

2. Cell culture medium: an Earle's balanced salt type minimum essential medium (MEM) additionally containing 0.1 M non-essential amino acids, 1.5 g/L of sodium bicarbonate, 0.1 M pyruvic acid, and 10% fetal bovine serum (from Gibco).

3. Phosphate buffered saline (PBS): purchased from Gibco with the product No. 10437-028.

4. DCFH-DA solution: a dye at a concentration of 5 μg/mL prepared from a fluorescent dye DCFH-DA (purchased from Sigma/SI-D6883-50 MG) in dimethyl sulfoxide (DMSO), which can be used for staining reactive oxygen species (ROS).

5. Trypsin (Trypsin-EDTA): 10× Trypsin-EDTA (purchased from Gibco) was diluted 10-fold in PBS.

6. Flow cytometer, BD Accuri C6 Plus.

### Test Process:

1. 2 mL of the cell culture medium was added to each well of a six-well culture plate and 2×10⁵ human skin fibrocytes were inoculated in each well.

2. The culture plate was placed in a CO₂ incubator and cultured at 37° C. for 24 hours to allow cell attachment.

3. After the cell attachment, the human skin fibrocytes were divided into the following three groups and subjected to corresponding treatment procedures.

Blank group: the medium in each well was replaced with 2 mL of pure cell culture medium (that is, not containing the peach extract of any embodiment), the cells were cultured at 5% CO₂ and 37° C. for 1 hour, then 2 μL of DCFH-DA solution was added to each well for reaction for 15 minutes, and then the cells were moved to a dark place for culture for another 15 minutes (at room temperature).

Control group: the medium in each well was replaced with 2 mL of pure cell culture medium (that is, not containing the peach extract of any embodiment), the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

Experimental group A: the medium in each well was replaced with a cell culture medium containing the peach extract (prepared in Example I) at a concentration of 0.0625 mg/mL, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

Experimental group B: the medium in each well was replaced with a cell culture medium containing the peach extract (prepared in Example I) at a concentration of 0.125 mg/mL, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

4. Then the cells of each group were rinsed twice with PBS.

5. Next, 200 μL of trypsin was added to each well to react in the absence of light for 5 minutes. After the reaction, the cell culture medium was added to each well to terminate the reaction.

6. The cells of each group were collected with the cell culture medium into individual corresponding centrifuge tubes, and the centrifuge tubes containing the cells and the cell culture medium were centrifuged at 400×g for 10 minutes.

7. After the centrifugation, a supernatant in each centrifuge tube was removed, and a cell precipitate in each centrifuge tube was washed with PBS.

8. Next, each centrifuge tube was centrifuged again at 400×g for 10 minutes.

9. After the re-centrifugation, a supernatant in each centrifuge tube was removed, and 1 mL of PBS was added to suspend a cell precipitate in each centrifuge tube to obtain a cell fluid to be tested.

10. The fluorescent signal of DCFH-DA in the cell fluid to be tested in each tube was detected by using a flow cytometer (BD Accuri C6 Plus Flow Cytometer 660517) to quantify the content of intracellular ROS. Herein, the excitation wavelength used for detecting the fluorescence signal was 450-490 nm, and the emission wavelength used therefor was 510-550 nm. After entering cells, DCFH-DA was firstly hydrolyzed into dichlorodihydrofluorescein (DCFH) and then oxidized by ROS into dichlorofluorescein (DCF) capable of emitting green fluorescence. The fluorescence intensity of the cells treated by the DCFH-DA can reflect the content of ROS in the cells. Therefore, the proportion of the number of the cells with the high expression of the ROS in the cells to the number of the original cells can be known.

### Test Results:

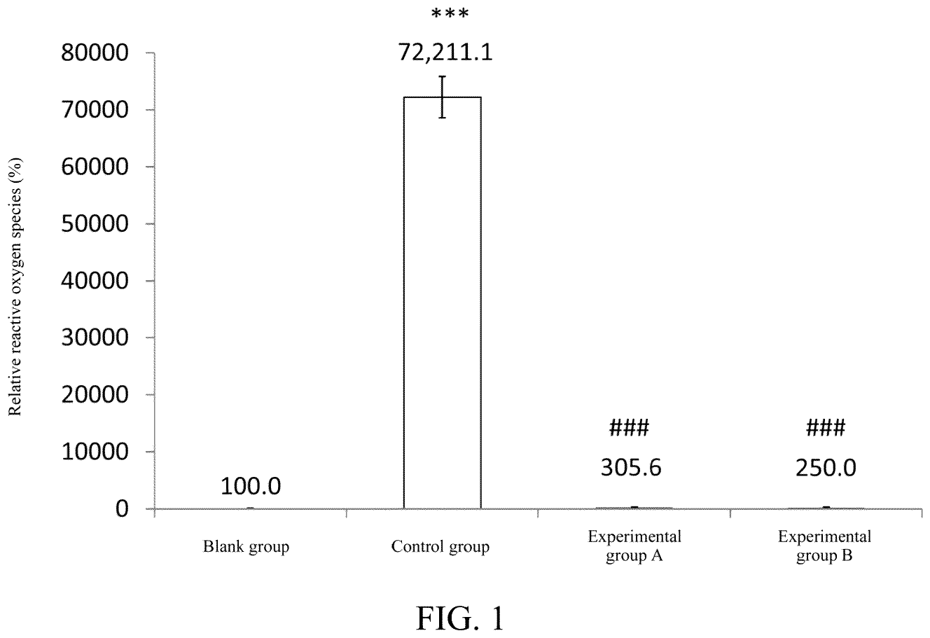

Herein, the human skin fibrocytes of the blank group were not irradiated with blue light. It also means that they grew under normal physiological metabolic conditions. Besides, when the content of ROS of the blank group was regarded as 100% of the relative ROS production amount, the content of ROS of each of the remaining groups was converted into the relative ROS production amount expressed in % by an interpolation method as shown in Table 1 and FIG. 1.

TABLE 1

| | Relative ROS production amount |
|---|---|
| Blank group | 100% |
| Control group | 72211.1% |
| Experimental group A | 305.6% |
| Experimental group B | 250.0% |

In FIG. 1, compared with the blank group, “*” represents a p value less than 0.05, “**” represents a p value less than 0.01, and “***” represents a p value less than 0.001; and compared with the control group, “#” represents a p value less than 0.05, “##” represents a p value less than 0.01, and “###” represents a p value less than 0.001. The more “*” or “#” represents the more statistically significant difference.

Refer to Table 1 and FIG. 1. After 15 minutes of the irradiation with blue light, the relative ROS production amount of human skin fibrocytes in the control group was up to 72211.1%. It can be known that the blue light can significantly promote the production of ROS by the human skin fibrocytes, which can greatly injure human skin.

Still refer to Table 1 and FIG. 1. After the human skin fibrocytes of the experimental group A were treated with the peach extract at a concentration of 0.0625 mg/mL, the relative ROS production amount was 305.6% even under the irradiation of blue light. Compared with the relative ROS production amount of the control group, the relative ROS production amount of the experimental group A was significantly reduced.

Still refer to Table 1 and FIG. 1. After the human skin fibrocytes of the experimental group B were treated with the peach extract at a concentration of 0.125 mg/mL, the relative ROS production amount was 250%. Compared with the relative ROS production amount of the control group, the relative ROS production amount of the test group B was significantly reduced.

On this basis, the test results show that the peach extract can significantly prevent the production of reactive oxygen species caused by blue light. In other words, the peach extract can be used to prevent the skin fibrocytes from being injured by the blue light irradiation. The peach extract can improve the resistance of the skin to the blue light and avoid the skin from being injured by the blue light irradiation.

### Example III Antioxidant Test

Reactive oxygen species (ROS) are one of the determinants for inducing free radical oxidative stress in vivo, which can damage and disable cells and promote cell apoptosis. The test determined whether or not it is possible to resist oxidative stress by measuring ROS. The oxidative stress can be caused by many factors such as chronic disease, stress anxiety, and overweight. Some external factors also cannot be ignored, such as pollution, smoking, drinking, drugs, sunlight, intense physical activity, or a sedentary lifestyle.

### Materials and Instruments:

1. Cell line: human skin fibroblasts CCD-966sk, obtained from the Bioresource Collection and Research Center (BCRC); Cat. 60153, hereinafter referred to as human skin fibrocytes.

2. Cell culture medium: a basic medium containing 10 vol % FBS (purchased from Gibco). The basic medium was prepared from an Eagle's minimal essential medium (MEM, purchased from Gibco, with the product No. 15188-319) additionally containing 1 mM sodium pyruvate (purchased from Gibco), 1.5 g/L of sodium bicarbonate (purchased from Sigma, Cat. S5761-500G), and 0.1 mM non-essential amino acid solution (purchased from Gibco).

3. Phosphate buffered saline (PBS): purchased from Gibco with the product No. 10437-028.

4. DCFH-DA solution: 2,7-dichloro-dihydro-fluorescein diacetate (DCFH-DA; product No. SI-D6883, purchased from Sigma) was dissolved in dimethyl sulfoxide (DMSO, purchased from Sigma, product No. SI-D6883-50 MG) to prepare 5 μg/mL of DCFH-DA solution.

5. Flow cytometer, BD Accuri C6 Plus.

6. Hydrogen peroxide (H₂O₂): purchased from Sigma-Aldrich, product model No. 95299-1L.

7. Trypsin (Trypsin-EDTA): 10× Trypsin-EDTA (purchased from Gibco) was diluted 10-fold in PBS.

### Test Process:

1. The human skin fibrocytes were inoculated at 1×10⁵ cells per well in a six-well culture plate containing 2 mL of the cell culture medium per well.

2. The culture plate was placed at 5% CO₂ and 37° C. for culture for 24 hours.

3. After the culture, the cells were divided into four groups, and the groups were subjected to different treatments.

Blank group: the medium in each well was replaced with 2 mL of pure cell culture medium (that is, not containing the peach extract of the present invention).

Control group: the medium in each well was replaced with 2 mL of pure cell culture medium.

Experimental group A: the medium in each well was replaced with 2 mL of cell culture medium containing the peach extract of Example I at a concentration of 0.0625 mg/mL.

Experimental group B: the medium in each well was replaced with 2 mL of cell culture medium containing the peach extract of Example I at a concentration of 0.125 mg/mL.

4. After the four groups were treated, the culture plate was placed at 5% CO₂ and 37° C. for 1 hour.

5. 2 μL of DCFH-DA solution was added to each well for reaction for 15 minutes.

6. H₂O₂ was added to react at 37° C. for 1 hour. In other words, 1 mM hydrogen peroxide was added to each group to simulate oxidative damage.

7. Then the cells in each well were rinsed twice with 1 mL of PBS.

8. 200 μL of trypsin was added to each well to react at a dark place for 5 minutes. After the reaction, the cell culture medium was added to each well to terminate the reaction.

9. The cells and the cell culture medium of each group were collected into individual corresponding centrifuge tubes, and the centrifuge tubes containing the cells and the cell culture medium were centrifuged at 400×g for 10 minutes.

10. After the centrifugation, a supernatant in each centrifuge tube was removed, and a cell precipitate in each centrifuge tube was redissolved with PBS.

11. Next, each centrifuge tube was centrifuged again at 400×g for 10 minutes.

12. After the re-centrifugation, a supernatant in each centrifuge tube was removed and a cell precipitate was suspended with 1 mL of PBS at a dark place to obtain a cell fluid to be tested.

13. The fluorescent signal of DCFH-DA in the cell fluid to be tested in each tube was detected by using a flow cytometer to quantify the content of intracellular ROS. The excitation wavelength used for detecting the fluorescence signal was 450-490 nm, and the emission wavelength used therefor was 510-550 nm. Since the test was performed in triplicate, the measurement results of the triplicate test in each group were averaged to obtain an average value as the content of ROS.

### Test Results:

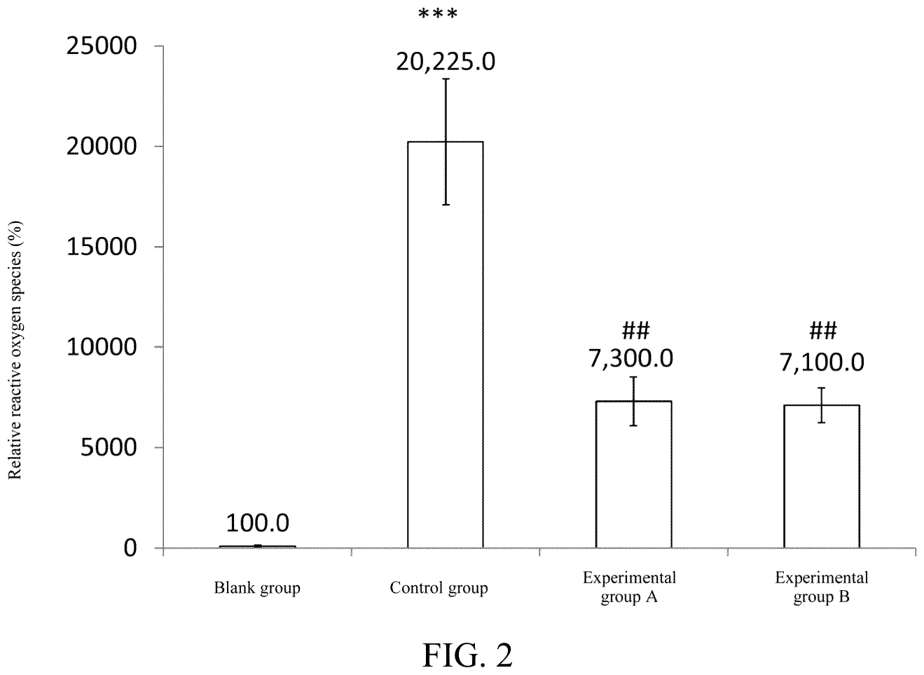

Herein, the human skin fibrocytes of the blank group were not subjected to damage by hydrogen peroxide. It also means that they were under normal physiological metabolic conditions. Therefore, it is assumed that its relative ROS production amount was 100%. Besides, when the content of ROS of the blank group was regarded as 100% of the relative ROS production amount, the content of ROS of each of the remaining groups was converted into the relative ROS production amount expressed in % by an interpolation method as shown in Table 2 and FIG. 2.

TABLE 2

| | Relative ROS production amount |
|---|---|
| Blank group | 100.0% |
| Control group | 20225.0% |
| Experimental group A | 7300.0% |
| Experimental group B | 7100.0% |

In FIG. 2, compared with the blank group, “*” represents a p value less than 0.05, “**” represents a p value less than 0.01, and “***” represents a p value less than 0.001; and compared with the control group, “#” represents a p value less than 0.05, “##” represents a p value less than 0.01, and “###” represents a p value less than 0.001. The more “*” or “#” represents the more statistically significant difference.

As shown in FIG. 2, by comparing the results of the blank group and the control group, it can be known that after the treatment with the hydrogen peroxide, the relative ROS production amount (high fluorescence expression) was greatly increased (to 20225%, statistically significant “***”, with a p value less than 0.001); and it indicates that the hydrogen peroxide treatment does promote the production of intracellular ROS, which can damage skin fibroblasts.

Still refer to Table 2 and FIG. 2. After the human skin fibrocytes of the experimental group A were treated with the peach extract at a concentration of 0.0625 mg/mL, the relative ROS production amount is reduced to 7300.0% even after the hydrogen peroxide treatment. Compared with the relative ROS production amount of the control group, the relative ROS production amount of the experimental group A is significantly reduced.

Still refer to Table 1 and FIG. 1. After the human skin fibrocytes of the experimental group B were treated with the peach extract at a concentration of 0.125 mg/mL, the relative ROS production amount was 7100.0%. Compared with the relative ROS production amount of the control group, the relative ROS production amount of the experimental group B was significantly reduced.

The above test results show that the peach extract of this example can effectively reduce the production or accumulation of ROS in the cells. In other words, the peach extract of this example can be used as a ROS scavenger. To be specific, the peach extract of this example can reduce the oxidative damage of the cells caused by ROS and the like by reducing the content of intracellular ROS. On this basis, the peach extract has an antioxidation function, prevents the skin from being damaged by the environment, maintains the normal function and structure of skin cells, reduces cell apoptosis caused by oxidative stress, and ensures healthier skin.

### Example IV Test for Promoting Production of Elastin

### Materials and Instruments:

1. Cell culture medium: a basic medium containing 10 vol % fetal bovine serum (FBS; trademark: Gibco). The basic medium was prepared from an Eagle's minimal essential medium (MEM, purchased from Gibco, with the product No. 15188-319) additionally containing 1 mM sodium pyruvate (trademark: Gibco).

2. Cell line: human skin fibroblasts CCD-966Sk (obtained from BCRC No. 60153), hereinafter referred to as human skin fibrocytes.

3. Human elastin detection kit (Human Elastin ELISA Kit, model ab239433, brand: Abcam).

4. Enzyme immunoanalyzer (brand: BioTek).

### Test Process:

1. The human skin fibrocytes were inoculated at 1×10⁴ cells per well in a 24-well culture plate containing 0.5 mL of the cell culture medium per well.

2. The culture plate was placed at 5% CO₂ and 37° C. for culture for 24 hours.

3. After the culture, the cells were divided into experimental groups and a blank group, which were correspondingly subjected to the following treatment procedures.

Blank group: the medium in each well was replaced with a pure cell culture medium (that is, a cell culture medium not containing the peach extract).

Experimental group A: the medium in each well was replaced with a cell culture medium containing the peach extract prepared in Example I at a concentration of 0.0625 mg/mL.

Experimental group B: the medium in each well was replaced with a cell culture medium containing the peach extract prepared in Example I at a concentration of 0.125 mg/mL.

4. After the treatment procedures, the culture plate was placed at 5% CO₂ and 37° C. for culture for 1 hour.

5. Next, after the human skin fibrocytes in each group were treated according to the standard process of the elastin detection kit, the production amount of elastin of the human skin fibrocytes in each group was detected by the enzyme immunoanalyzer.

### Test Results:

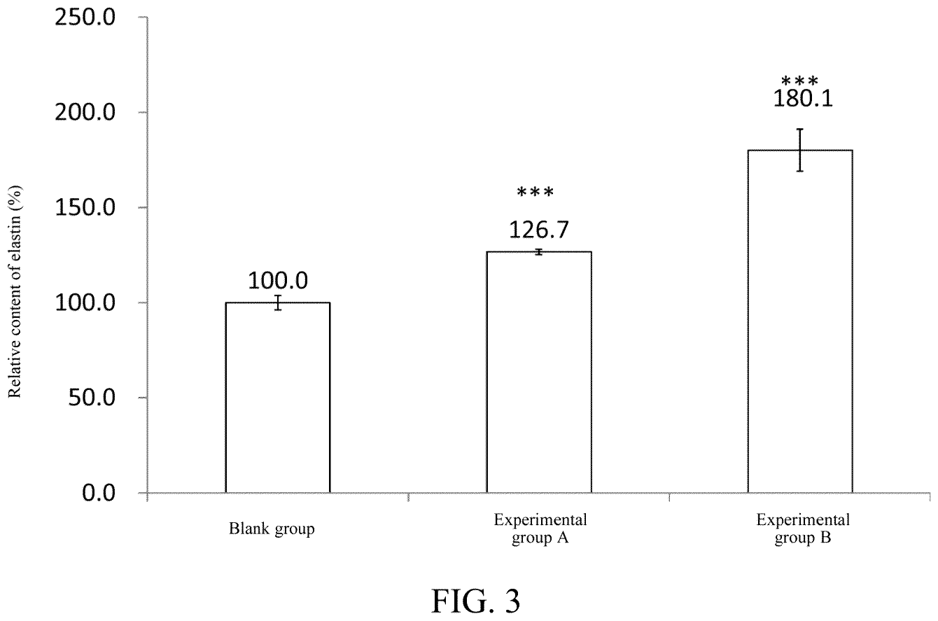

Herein, the content of elastin measured in the blank group was regarded as 100% (that is, the relative production amount of elastin under normal physiological metabolic conditions was 100%), and the content of elastin measured in the experimental groups was correspondingly converted into the production amount of elastin in %, as shown in FIG. 3. In FIG. 3, “*” represents a p value less than 0.05, “*” represents a p value less than 0.01, and “***” represents a p value less than 0.001. The more “*” represents the more statistically significant difference.

Referring to FIG. 3, on the basis that the production amount of elastin in the blank group was 100%, the production amount of elastin in the experimental group A was 126.7%. The production amount of elastin in the experimental group was significantly increased by 26.7%, which represents that the human skin fibrocytes treated by the peach extract for 24 hours can produce more elastin.

Still referring to FIG. 3, on the basis that the production amount of elastin in the blank group was 100%, the production amount of elastin in the experimental group B was 180.1%. The production amount of elastin in the experimental group was significantly increased by 80.1%, which represents that the human skin fibrocytes treated by the peach extract for 24 hours can produce more elastin.

Therefore, the peach extract of this example has the function of promoting the cells to synthesize elastin. On this basis, the peach extract has the function of promoting the cells to synthesize elastin. After the cells are treated by the peach extract, the skin cells can be promoted to produce elastin, thereby improving skin elasticity.

### Example V Test for Activity of Mitochondria

Mitochondria are the key organelles of cells producing energy and regulate cell metabolism and survival through oxidative phosphorylation (or electron transport chains). Mitochondrial dysfunction can lead to abnormal proliferation of ROS, possibly further causing genomic (mtDNA) mutation of mitochondria themselves, cell apoptosis and cell damage. In the present test, a JC-1 dye is used for observing the mitochondrial membrane potential of human skin cells. When the mitochondrial membrane potential is increased, JC-1 can be aggregated by negative electricity on a mitochondrial inner membrane to form a polymer, namely, when the JC-1 aggregation amount is high, the activity of mitochondria in the skin cells can be regarded as being improved, representing healthier skin cells.

### Materials and Instruments:

1. Cell line: human skin fibroblasts CCD-966sk (BCRC No. 60153).

2. Cell culture medium: a basic medium containing 10 vol % FBS (purchased from Gibco). The basic medium was prepared from an Eagle's minimal essential medium (MEM, purchased from Gibco, with the product No. 15188-319) additionally containing 1 mM sodium pyruvate (purchased from Gibco), 1.5 g/L of sodium bicarbonate (purchased from Sigma), and 0.1 mM non-essential amino acid solution (purchased from Gibco).

3. Phosphate buffered saline (PBS): purchased from Gibco.

4. Mitochondrial membrane potential detection kit (BDTM MitoScreen (JC-1) kit, model 551302). The mitochondrial membrane potential detection kit includes a JC-1 dye (freeze-dried) and a 10× analysis buffer. Prior to use, the 10× analysis buffer was diluted 10-fold with PBS to form a 1× analysis buffer. 130 μL of DMSO was added to the JC-1 dye (freeze-dried) to form a JC-1 stock solution. Then the JC-1 stock solution was diluted with the 1× analysis buffer to form a JC-1 working solution (a JC-1 mitochondrial specific dye). A dilution ratio of the JC-1 stock solution to the 1× analysis buffer was 1:100.

5. Trypsin: 10× Trypsin-EDTA (purchased from Gibco) was diluted 10-fold in 1×PBS.

6. Flow cytometer: purchased from the BD Pharmingen company, model BDTM Accuri C6 Plus.

7. Peach extract: the peach extract obtained by the preparation method of Example I of the present invention.

### Test Steps:

1. The human skin fibrocytes were inoculated at 1×10⁵ cells per well in a six-well culture plate containing 2 mL of the cell culture medium per well.

2. The culture medium in each well of the plate was replaced with 2 mL of experimental culture medium. The experimental culture medium of an experimental group A was a cell culture medium containing 0.0625 mg/mL of the peach extract, the experimental culture medium of an experimental group B was a cell culture medium containing 0.125 mg/mL of the peach extract, and the experimental culture medium of a blank group was a pure cell culture medium (not containing the peach extract).

3. The culture plate was placed at 5% CO₂ and 37° C. for culture for 24 hours.

4. The experimental culture medium in the culture plate was removed and rinsed twice with 1 mL of PBS.

5. 200 μL of trypsin was added to each well to react at a dark place for 5 minutes. After the reaction, the cell culture medium was added to terminate the reaction. The cells and the cell culture medium in each well were collected into individual corresponding centrifuge tubes, and the centrifuge tubes containing the cells and the cell culture medium were centrifuged at 400×g for 10 minutes.

6. A supernatant was removed after the centrifugation, a cell precipitate was redissolved with 1 mL of PBS or transferred to another centrifuge tube to obtain the centrifuge tube containing a cell suspension.

7. The centrifuge tube containing the cell suspension was centrifuged at 400×g for 5 minutes.

8. After the centrifugation, a supernatant in each centrifuge tube was removed and 100 μL of JC-1 working solution was added to each centrifuge tube.

9. The cell precipitate in each centrifuge tube was vortexed uniformly with the JC-1 working solution and cultured at a dark place for 15 minutes.

10. After 15 minutes, each centrifuge tube was centrifuged at 400×g for 5 minutes.

11. After the centrifugation, a supernatant in each centrifuge tube was removed, and a cell precipitate in each centrifuge tube was redissolved with 1 mL of PBS and centrifuged at 400×g for 5 minutes.

12. After the centrifugation, a supernatant in each centrifuge tube was removed, and a cell precipitate in each centrifuge tube was redissolved with 1 mL of PBS and centrifuged at 400×g for 5 minutes.

13. After the centrifugation, a supernatant in each centrifuge tube was removed and the cells were resuspended with 500 μL of PBS to obtain a cell fluid to be tested.

14. The membrane potential of cell mitochondria in the cell fluid to be tested in each well was measured by using the flow cytometer to analyze the activity of mitochondria.

### Test Results:

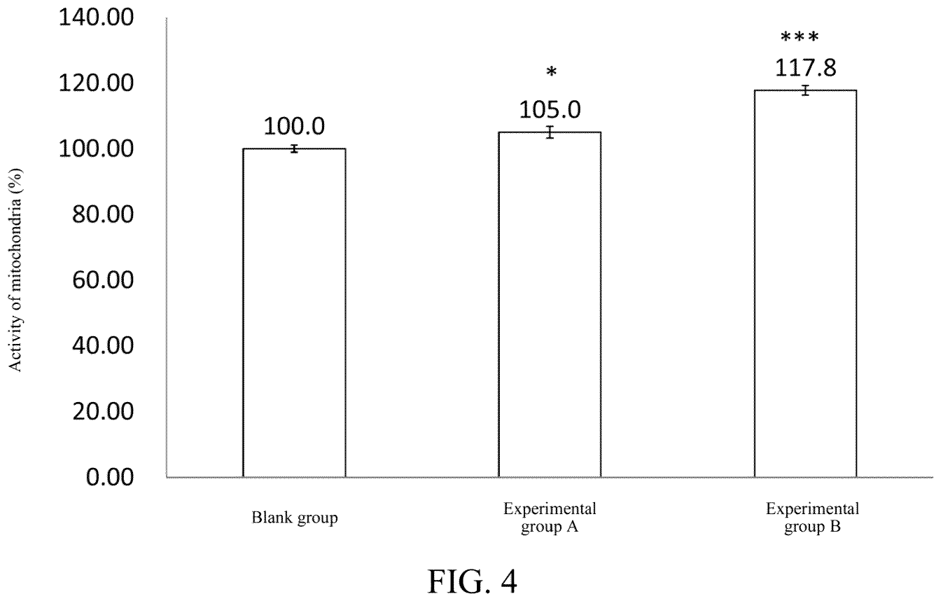

Herein, the test result of the blank group was regarded as 100% (that is, the relative JC-1 aggregation amount was 100%), and the test results of the experimental groups were correspondingly converted to the relative JC-1 aggregation amount in % as shown in FIG. 4. When the JC-1 aggregation amount is high, the cells were more active, and when JC-1 aggregation amount was low, the cells were closer to the apoptotic state.

Referring to FIG. 4, the relative JC-1 aggregation amount of the experimental group A was about 105.0%. In other words, compared with the blank group, the activity of mitochondria of the human skin fibroblasts in the experimental group A was increased by 5%. It indicates that the peach extract can increase the activity of mitochondria of the skin cells.

Still referring to FIG. 4, the relative JC-1 aggregation amount of the experimental group B was about 117.8%. In other words, compared with the blank group, the activity of mitochondria of the human skin fibroblasts in the experimental group B was greatly increased by 17.8%. It indicates that the peach extract can increase the activity of mitochondria of the skin cells.

On this basis, the peach extract has the function of promoting the activity of intracellular mitochondria. After the cells were treated by the peach extract, the apoptosis of mitochondria in the skin cells can be avoided, such that the mitochondria can provide more sufficient energy for the cells. Simultaneously, gene mutation or cell apoptosis caused by abnormal proliferation of ROS can be avoided, and the production of free radicals in the cells can be reduced.

### Example VI Human Body Test

Subjects: seven subjects (adults with self-reported poor complexion or loose skin and aged from 20 to 55 years old).

### Test Items and Instruments

1. Skin wrinkle amount: a VISIA high-order digital skin detector sold by Canfield in the United States was used for detection. The facial skin, especially the periocular region of the same subject before and after drinking was photographed by using a high-resolution camera lens. The texture position can be detected by detecting the changes of skin fine lines through standard white light irradiation to obtain a value, representing the smoothness of the skin. After the measurement, with the expression amount of the wrinkle amount of a blank group as a baseline (that is, the relative expression rate of the wrinkle amount of the blank group was 100%), the relative expression rate (%) of the skin wrinkle amount of an experimental group was calculated.

2. Skin texture: the VISIA high-order digital skin detector sold by Canfield in the United States was used for detecting the facial skin of the subjects. The principle is to take a high-resolution skin image with a visible light and perform roughness analysis with a built-in software according to the pits and projections of the skin to obtain a skin texture value. A higher skin texture measured value indicates rougher skin. After the measurement, with the expression amount of the skin texture of the blank group as a baseline (that is, the relative expression rate of the skin texture of the blank group was 100%), the relative expression rate (%) of the skin texture of the experimental group was calculated.

3. Trichopore: the VISIA high-order digital skin detector sold by Canfield in the United States was used for detecting the facial skin of the subjects. The principle is to measure the expression amount of each group of trichopores in the facial skin of the subjects through the high-resolution camera lens, and generate the shadow at the pitted part of the facial trichopores by the standard white light irradiation, such that the trichopores are darker than the surrounding skin, and the number and the area of the trichopores can be detected. Then the numerical trichopore expression amount is obtained by analyzing the number and the area of the trichopores by using a software. The higher numerical value indicates more trichopore number and larger area. After the measurement, with the expression amount of the trichopores of the blank group as a baseline (that is, the relative expression rate of the trichopores in the skin of the blank group was 100%), the relative expression rate (%) of the trichopores in the skin of the experimental group was calculated.

4. Content of heme and content of iron in blood: entrusted to the Lezen Medical Laboratory for the detection. Herein, the higher content of heme and content of iron in blood can increase oxygen metabolism and relieve anemia, thereby further increasing the oxygen content of the skin cells to promote skin repair. It can also be observed that the skin was ruddier and glossier, namely the appearance was better.

### Test Process:

A 50-mL bottle of peach sample containing 0.9 g of the peach extract prepared in Example I was administered to the subjects per day for successive four weeks. Besides, blood was collected from each subject before the first administration (that is, the blank group) and after four weeks of administration (that is, the experimental group). The facial skin of the subjects was detected by using the digital skin detector.

Herein, each bottle of the peach sample includes 0.9 g of the peach extract prepared in Example I, 0.05 g of citric acid, 0.0075 g of stevioside, 0.05 g of juicy peach liquid flavor A, 0.05 g of juicy peach liquid flavor B, and 48.9425 g of water.

### Test Results:

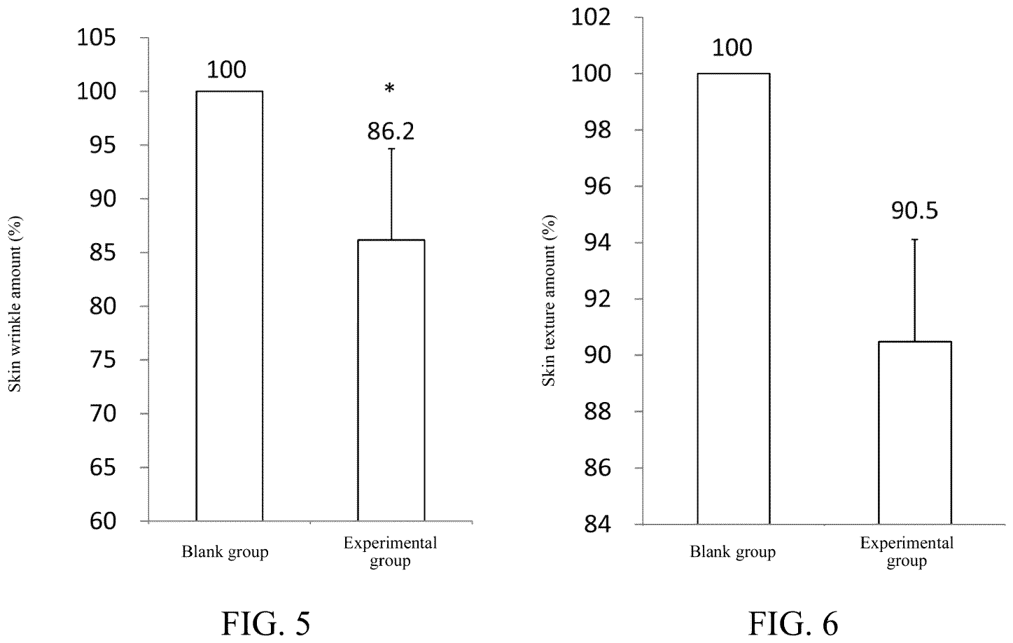

It can be seen from FIG. 5 that, in the case where the relative expression rate of the skin wrinkle amount of the blank group was 100%, the relative expression rate of the skin wrinkle amount of the experimental group was 86.2%, meaning that the wrinkle amount on the facial skin of the subjects was reduced after the subjects continuously drunk the peach extract for four weeks compared to that before use. In other words, the peach extract can reduce wrinkles on the skin, increase skin flatness, relieve the aging state of the skin, and thus make the skin more delicate and glossier.

It can be seen from FIG. 6 that, in the case where the relative expression rate of the skin texture amount of the blank group was 100%, the relative expression rate of the skin texture amount of the experimental group was 90.5%, meaning that the skin texture on the facial skin of the subjects was reduced after the subjects continuously drunk the peach extract for four weeks compared to that before use. In other words, the peach extract can improve skin texture, so as to relieve rough skin, increase skin flatness, and further make the skin more delicate and glossier.

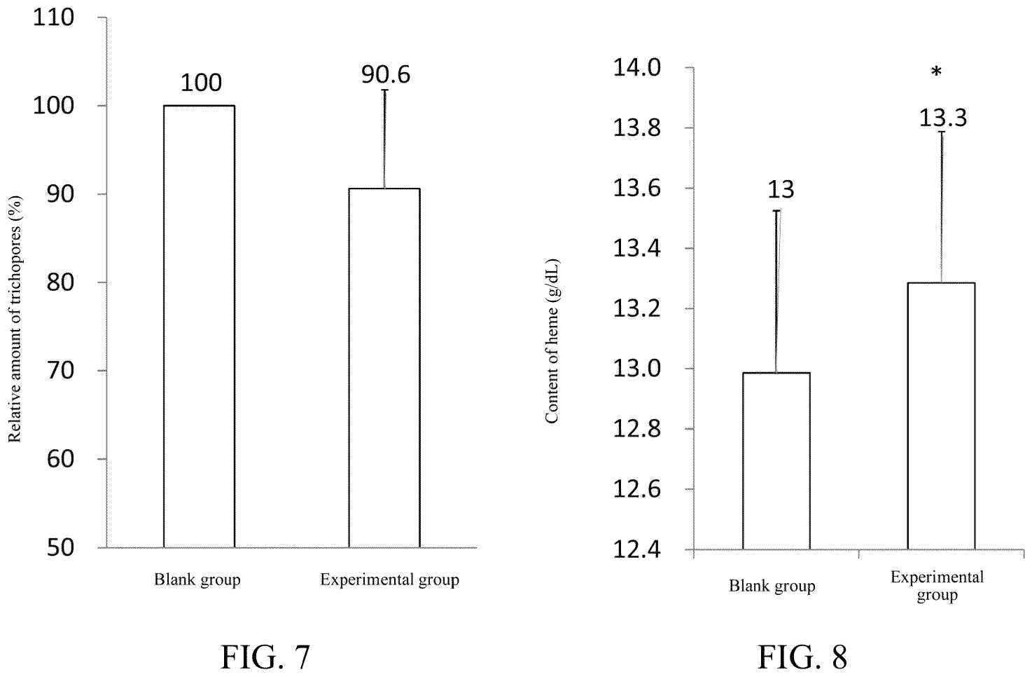

It can be seen from FIG. 7 that, in the case where the relative expression rate of the trichopores of the blank group was 100%, the relative expression rate of the trichopores of the experimental group was 90.6%, meaning that the trichopores in the facial skin of the subjects were reduced after the subjects continuously drunk the peach extract for four weeks compared to that before use. In other words, the peach extract can minimize the trichopores in the skin, so as to relieve the rough skin, tighten the trichopores, and further make the skin more delicate and glossier.

Referring to FIG. 8, in the case where the content of heme of the blank group was 13 g/dL, the content of heme of the experimental group was 13.3 g/dL. It can be known that compared with that before the drinking, the content of heme in blood is significantly increased by 2.3% after the subjects continuously drunk the peach extract for four weeks. In other words, the peach extract can increase the content of heme in human body blood.

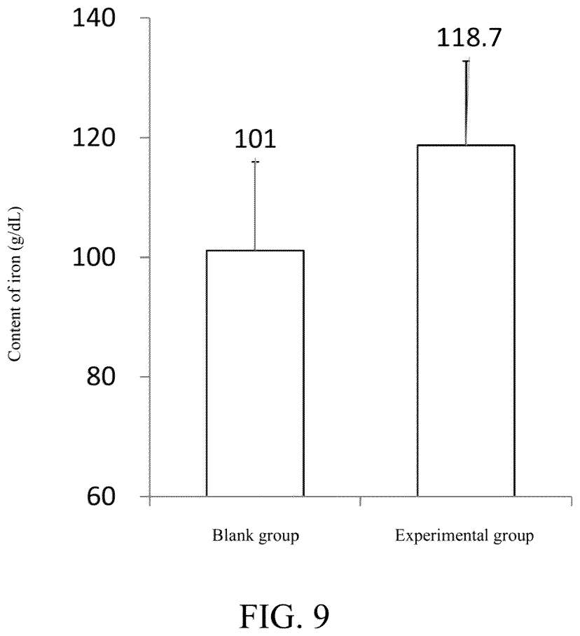

Referring to FIG. 9, in the case where the content of iron of the blank group was 101 g/dL, the content of iron of the experimental group was 118.7 g/dL. It can be known that compared with that before the drinking, the content of iron in blood was significantly increased by 17.4% after the subjects continuously drunk the peach extract for four weeks. In other words, the peach extract can increase the content of iron in human body blood, so as to increase oxygen metabolism and relieve anemia, thereby further increasing the oxygen content of the skin cells to promote skin repair. It can also be observed that the skin was ruddier and glossier, namely the appearance was better.

### Example VII Component Analysis of Bioactive Substances of Peach Extract

Extracts of natural plants generally contain a variety of components and are not pure substances. The solubility of different bioactive substances in different solvents is different. In the test, mutually immiscible solvents are utilized to transfer a specific component in the peach extract to another solvent.

### Instruments:

(1) Nuclear magnetic resonance spectrometer (NMR). 1D and 2D spectra were expressed in chemical shifts in 8 with the unit of ppm by using Ascend 400 MHZ, Bruker Co., Germany.

(2) Mass spectrometer (MS) tandem mass spectrometer-two-dimensional ion trap tandem Fourier transform mass spectrometer and ESI-MS/MS: measured by using a Bruker amaZon SL system with the unit of m/z.

(3) Medium pressure liquid chromatography (MPLC): CombiFlash® Rf+, Teledyne ISCO, Lincoln, NE; high performance liquid chromatography (HPLC): high performance liquid chromatography (HPLC) was Agilent 1200 series; a degasser was an Agilent vacuum degasser 1322A; an extraction solvent delivery system was Agilent quaternary pump G1311A; a multiple wavelength detector (MWD) Agilent G1314B; and a diode array detector (DAD) was Agilent 1260 Infinity DAD VL G1315D with the detection wavelengths of 210 nm, 280 nm, 320 nm and 365 nm (Agilent Germany).

(4) Analysis column: Luna®5 μm C18 (2) 100 Å (250×10 mm, Phenomenex, USA).

(5) Column chromatography packing material: Sephadex LH-20 (Pharmacia, Piscataway, NJ, USA), macroporous resin Diaion HP-20 (Mitsubishi Chemical Co., Japan), normal phase silica gel Merck Kieselgel 60 (40-63 μm, Art. 9385), and reverse phase silica gel Merck LiChroprep® RP-18 (40-63 μm, Art. 0250).

(6) Thin-layer chromatography using TLC aluminum sheets (Silica gel 60 F254, 0.25 mm, Merck, Germany) and TLC aluminum sheets (RP-18F 254-S, 0.25 mm, Merck, Germany).

(7) Solvents: n-hexane, ethyl acetate, acetone, methanol, ethanol, acetonitrile (purchased to Merck, Taiwan), chloroform-d1 (deuteration degree 99.5%), methanol-d4 (deuteration degree 99.5%), deuterium oxide (deuteration degree >99.8%), and dimethyl sulfoxide-d6 (deuteration degree >99.9%) (Merck, Taiwan).

### Test Process:

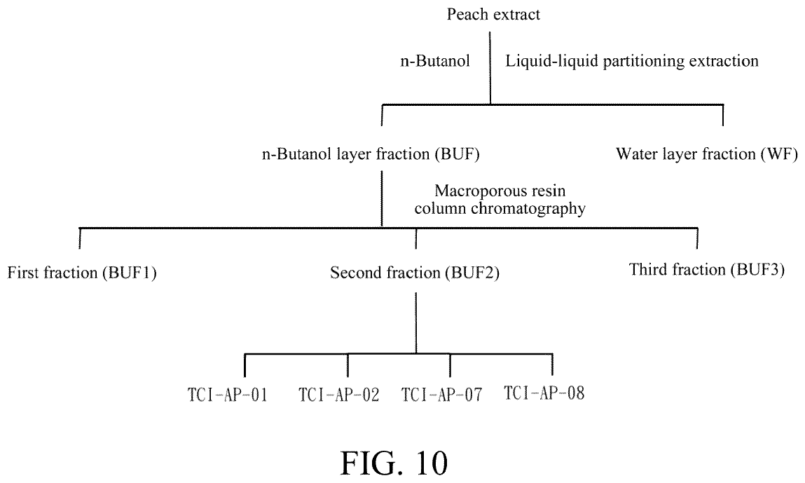

Refer to FIG. 10. Firstly, 5 liters (L) of the peach extract prepared in Example I was taken and separated by liquid phase partitioning with n-butanol and water in equal proportions to respectively obtain an n-butanol layer extract and a water layer extract. Then, the n-butanol layer extract was concentrated under reduced pressure and dried to obtain 34.2 g of n-butanol layer fraction (BUF). The water layer extract was concentrated under reduced pressure and dried to obtain 267.1 g of water layer fraction (WF).

Bioassay guided fractionation was subsequently used, Sephadex LH-20 column chromatography was performed on the n-butanol layer fraction by using methanol as an eluent, thin-layer chromatography was subsequently used, and eluates with similar results were combined to obtain a first fraction (BUF1), a second fraction (BUF2), and a third fraction (BUF3).

Still refer to FIG. 10. The second fraction (BUF2) was subjected to re-separation by a reverse phase-medium pressure liquid chromatography (RP-MPLC) to obtain a plurality of eluates. Herein, linear elution was performed by water to methanol at a flow rate of 10 mL/min for 60 minutes. The eluates with similar results were combined subsequently by using thin layer chromatography (TLC aluminum sheet: thin layer chromatography sheet, silica gel-coated 60 F254 (0.25 mm)) to obtain a plurality of sub-fractions including a BUF2-2 fraction, a BUF2-5 fraction, a BUF2-8 fraction and a BUF2-9 fraction.

The BUF2-2 fraction was purified by reverse-high efficiency liquid chromatography (volume ratio: methanol/water=2/3) to obtain a bioactive substance TCI-AP-01. After analyzing its chemical structure by hydrogen-nuclear magnetic resonance spectroscopy (1H-NMR) and electrospray ionization mass spectrometry (ESIMS), the bioactive substance was determined to be hyperoside, whose IUPAC name was 2-(3,4-dihydroxyphenyl)-5,7-dihydroxy-3-[(2S,3R,4S,5R,6R)-3,4,5-trihydroxy-6-(hydroxymethyl) oxan-2-yl]oxychromen-4-one and whose chemical structural formula was as follows:

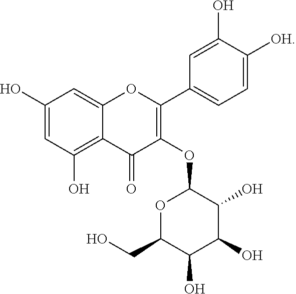 [SMILES: O=c1c(O[C@@H]2O[C@H](CO)[C@H](O)[C@H](O)[C@H]2O)c(-c2ccc(O)c(O)c2)oc2cc(O)cc(O)c12]

The BUF2-5 fraction was purified by reverse-high efficiency liquid chromatography (volume ratio: methanol/water=1/3) to obtain a bioactive substance TCI-AP-02. After analyzing its chemical structure by hydrogen-nuclear magnetic resonance spectroscopy (1H-NMR) and electrospray ionization mass spectrometry (ESIMS), the bioactive substance was determined to be isoquercitrin, whose IUPAC name was 2-(3,4-dihydroxyphenyl)-5,7-dihydroxy-3-[(2S,3R,4S,5S,6R)-3,4,5-trihydroxy-6-(hydroxymethyl) oxan-2-yl]oxychromen-4-one and whose chemical structural formula was as follows:

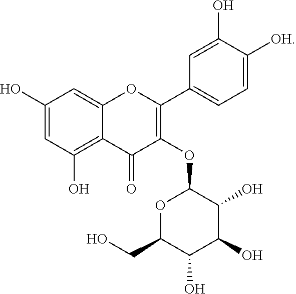 [SMILES: O=c1c(O[C@@H]2O[C@H](CO)[C@@H](O)[C@H](O)[C@H]2O)c(-c2ccc(O)c(O)c2)oc2cc(O)cc(O)c12]

The BUF2-8 fraction was purified by reverse-high efficiency liquid chromatography (volume ratio: methanol/water-2/5) to obtain a bioactive substance TCI-AP-07. After analyzing its chemical structure by hydrogen-nuclear magnetic resonance spectroscopy (1H-NMR) and electrospray ionization mass spectrometry (ESIMS), the bioactive substance was determined to be nicotiflorin, whose IUPAC name was 5,7-dihydroxy-2-(4-hydroxyphenyl)-3-[(2S,3R,4S,5S,6R)-3,4,5-trihydroxy-6-[[(2R,3R,4R,5R,6S)-3,4,5-trihydroxy-6-methyloxan-2-yl]oxymethyl]oxan-2-yl]oxychromen-4-one and whose chemical structural formula was as follows:

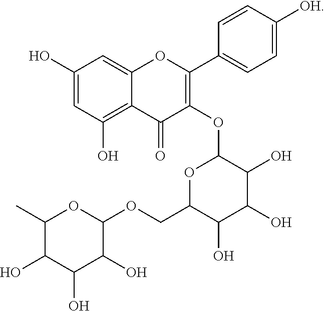 [SMILES: CC1OC(OCC2OC(Oc3c(-c4ccc(O)cc4)oc4cc(O)cc(O)c4c3=O)C(O)C(O)C2O)C(O)C(O)C1O]

The BUF2-9 fraction was purified by reverse-high efficiency liquid chromatography (volume ratio: methanol/water-3/4) to obtain a bioactive substance TCI-AP-08. After analyzing its chemical structure by hydrogen-nuclear magnetic resonance spectroscopy (1H-NMR) and electrospray ionization mass spectrometry (ESIMS), the bioactive substance was determined to be rutin, whose IUPAC name was butyl (1S,3R,4R,5R)-3-[(E)-3-(3,4-dihydroxyphenyl) prop-2-enoyl]oxy-1,4,5-trihydroxycyclohexane-1-carboxylate and whose chemical structural formula was as follows:

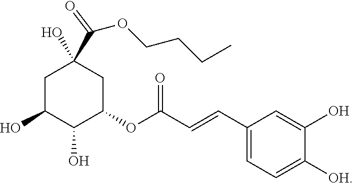 [SMILES: CCCCOC(=O)[C@@]1(O)C[C@H](OC(=O)/C=C/c2ccc(O)c(O)c2)[C@H](O)[C@@H](O)C1]

It can be known that the peach extract contains at least bioactive substances such as hyperoside (TCI-AP-01), isoquercitrin (TCI-AP-02), nicotiflorin (TCI-AP-07), and rutin (TCI-AP-08).

### Example VIII Fingerprint Analysis of Peach Extract

Herein, the bioactive substances in the peach extract prepared in Example I were quantitatively and qualitatively analyzed by high performance liquid chromatography (HPLC).

### Test Process:

The peach extract prepared in Example I was used as a sample, the concentration was 50 mg/mL, and the injection volume was 10 μL.

The solvents used were methanol and water, 0.1% formic acid was added to each of methanol and water, and the flow rate was set to be 1 mL/min. The elution condition was set as follows: methanol:water was 2:98 at 0 minute, methanol:water was 2:98 at 10 minutes, methanol:water was 70:30 at 40 minutes, methanol:water was 100:0 at 50 minutes, and methanol:water was 100:0 at 60 minutes. The column temperature was set to be 40° C.

### Test Results:

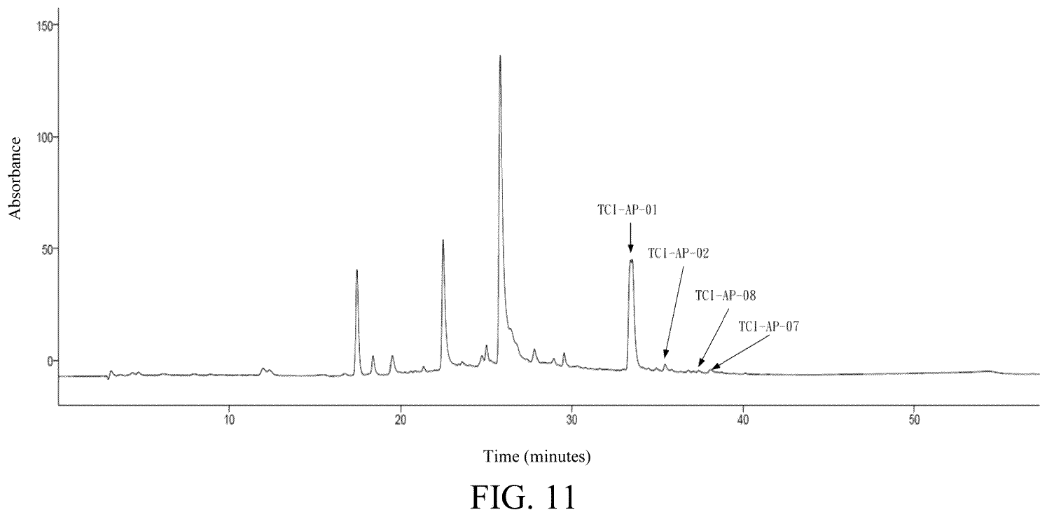

Referring to FIG. 11, the wave peak of the bioactive substance TCI-AP-01 was resolved at around a time of 34 minutes, and the wave peaks of the bioactive substance TCI-AP-02, the bioactive substance TCI-AP-08, and the bioactive substance TCI-AP-07 were resolved sequentially at between a time of 35 and 40 minutes.

### Example IX Blue Light-Induced Injury Resistance Test of Bioactive Substances

### Materials and Instruments:

1. Cell line: human skin fibroblasts CCD-966sk, obtained from the Bioresource Collection and Research Center (BCRC); Cat. 60153, hereinafter referred to as human skin fibrocytes.

2. Cell culture medium: an Earle's balanced salt type minimum essential medium (MEM) additionally containing 0.1 M non-essential amino acids, 1.5 g/L of sodium bicarbonate, 0.1 M pyruvic acid, and 10% fetal bovine serum (from Gibco).

3. Phosphate buffered saline (PBS): purchased from Gibco with the product No. 10437-028.

4. DCFH-DA solution: a dye at a concentration of 5 mg/mL prepared from a fluorescent dye DCFH-DA (purchased from Sigma/SI-D6883-50 MG) in dimethyl sulfoxide (DMSO), which can be used for staining reactive oxygen species (ROS).

5. Trypsin (Trypsin-EDTA): 10× Trypsin-EDTA (purchased from Gibco) was diluted 10-fold in PBS.

6. Flow cytometer, BD Accuri C6 Plus.

### Test Process:

The room temperature for the test was 25±5° C.

1. 2 mL of the cell culture medium was added to each well of a six-well culture plate and 1×10⁵ human skin fibrocytes CCD-966sk (preservation No. BCRC 60153) were inoculated in each well.

2. The culture plate was placed at 5% CO₂ and 37° C. for culture for 24 hours.

3. After the culture, the cells were divided into the following three groups and subjected to corresponding treatment procedures.

Blank group: the medium in each well was replaced with 2 mL of pure cell culture medium (that is, not containing the peach extract of the present invention), the cells were cultured at 5% CO₂ and 37° C. for 1 hour, then 2 μL of DCFH-DA solution was added to each well for reaction for 15 minutes, and then the cells were moved to a dark place for culture for another 15 minutes (at room temperature).

Control group: the medium in each well was replaced with 2 mL of pure cell culture medium (that is, not containing the peach extract of the present invention), the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

TCI-AP-01 group: the medium in each well was replaced with a cell culture medium containing hyperoside (separated from Example VII) at a concentration of 0.1 mM, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

TCI-AP-02 group: the medium in each well was replaced with a cell culture medium containing isoquercitrin (separated from Example VII) at a concentration of 0.1 mM, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

TCI-AP-07 group: the medium in each well was replaced with a cell culture medium containing nicotiflorin (separated from Example VII) at a concentration of 0.1 mM, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

TCI-AP-08 group: the medium in each well was replaced with a cell culture medium containing rutin (separated from Example VII) at a concentration of 0.1 mM, the cells were cultured at 5% CO₂ and 37° C. for 1 hour, then 2 μL of DCFH-DA solution was added to each well for reaction for 15 minutes, and then the cells were transferred to a blue light box and irradiated with blue light (with a wavelength of 500 nm) for 15 minutes (at room temperature).

4. Then, the cells of each group were rinsed twice with PBS.

5. Next, 200 μL of trypsin was added to each well to react in the absence of light for 5 minutes. After the reaction, the cell culture medium was added to each well to terminate the reaction.

6. The cells of each group were collected with the cell culture medium into individual corresponding centrifuge tubes, and the centrifuge tubes containing the cells and the cell culture medium were centrifuged at 400×g for 10 minutes.

7. After the centrifugation, a supernatant in each centrifuge tube was removed, and a cell precipitate in each centrifuge tube was washed with PBS.

8. Next, each centrifuge tube was centrifuged again at 400×g for 10 minutes.

9. After the re-centrifugation, a supernatant in each centrifuge tube was removed, and 1 mL of PBS was added to suspend a cell precipitate in each centrifuge tube to obtain a cell fluid to be tested.

10. The fluorescent signal of DCFH-DA in the cell fluid to be tested in each tube was detected by using a flow cytometer (BD Accuri C6 Plus Flow Cytometer 660517) to quantify the content of intracellular ROS. Herein, the excitation wavelength used for detecting the fluorescence signal was 450-490 nm, and the emission wavelength used therefor was 510-550 nm. After entering cells, DCFH-DA was firstly hydrolyzed into dichlorodihydrofluorescein (DCFH) and then oxidized by ROS into dichlorofluorescein (DCF) capable of emitting green fluorescence. The fluorescence intensity of the cells treated by the DCFH-DA can reflect the content of ROS in the cells. Therefore, the proportion of the number of the cells with the high expression of the ROS in the cells to the number of the original cells can be known. Herein, each group was subjected to the test in triplicate and the average value of the measurement results of the triplicate test was rounded to be the content of ROS.

### Test Results:

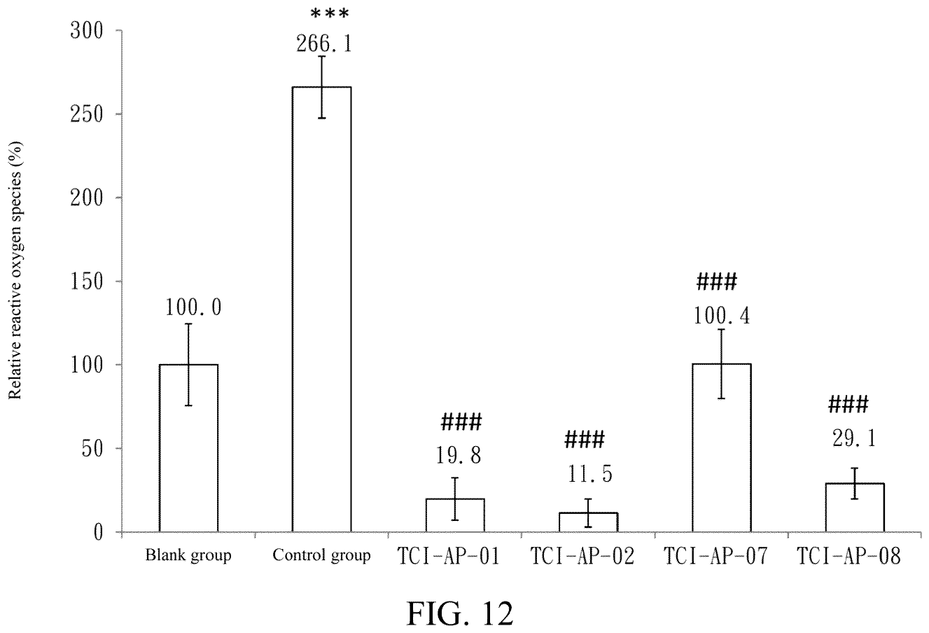

Herein, the human skin fibrocytes of the blank group were not irradiated with blue light. It also means that they were under normal physiological metabolic conditions. Therefore, it is assumed that its relative ROS production amount was 100%. Besides, when the content of ROS of the blank group was regarded as 100% of the relative ROS production amount, the content of ROS of each of the remaining groups was converted into the relative ROS production amount expressed in % by an interpolation method as shown in Table 3 and FIG. 12.

TABLE 3

| | Relative ROS production amount |
|---|---|
| Blank group | 100% |
| Control group | 266.1% |
| TCI-AP-01 | 19.8% |
| TCI-AP-02 | 11.5% |
| TCI-AP-07 | 100.4% |
| TCI-AP-08 | 29.1% |

In FIG. 12, compared with the blank group, “*” represents a p value less than 0.05, “**” represents a p value less than 0.01, and “***” represents a p value less than 0.001; and compared with the control group, “#” represents a p value less than 0.05, “##” represents a p value less than 0.01, and “###” represents a p value less than 0.001. The more “*” or “#” represents the more statistically significant difference.

Refer to Table 3 and FIG. 12. After 15 minutes of the irradiation with blue light, the relative ROS production amount of human skin fibrocytes in the control group was up to 266.1%. It can be known that the blue light can significantly promote the production of ROS by the human skin fibrocytes, thereby greatly injuring human skin.

After the human skin fibrocytes of the TCI-AP-01 group are treated with the hyperoside, the relative ROS production amount was 19.8% even under the irradiation of blue light. Compared with the relative ROS production amount of the control group or the blank group, the relative ROS production amount of the TCI-AP-01 group was significantly reduced.

After the human skin fibrocytes of the TCI-AP-02 group are treated with the isoquercitrin, the relative ROS production amount was 11.5% even under the irradiation of blue light. Compared with the relative ROS production amount of the control group or the blank group, the relative ROS production amount of the TCI-AP-02 group was significantly reduced.

After the human skin fibrocytes of the TCI-AP-07 group are treated with the nicotiflorin, the relative ROS production amount was 100.4% even under the irradiation of blue light. The relative ROS production amount of the TCI-AP-07 group was significantly reduced compared with the relative ROS production amount oof the control group and almost the same compared with the relative ROS production amount of the blank group. In other words, the injury caused by the blue light can be almost cancelled after the treatment with the nicotiflorin.

After the human skin fibrocytes of the TCI-AP-08 group are treated with the rutin, the relative ROS production amount was 29.1% even under the irradiation of blue light. Compared with the relative ROS production amount of the control group or the blank group, the relative ROS production amount of the TCI-AP-08 group was significantly reduced.

On this basis, the test results show that the bioactive substances in the peach extract can significantly prevent the production of reactive oxygen species caused by blue light.

### Example X Antioxidant Test of Bioactive Substances

Reactive oxygen species (ROS) are one of the determinants for inducing free radical oxidative stress in vivo. The test determines whether or not it is possible to resist oxidative stress by measuring ROS. The oxidative stress can be caused by many factors such as chronic disease, stress anxiety, and overweight. Some external factors also cannot be ignored, such as pollution, smoking, drinking, drugs, sunlight, intense physical activity, or a sedentary lifestyle.

### Materials and Instruments:

1. Cell line: human skin fibroblasts CCD-966sk, obtained from the Bioresource Collection and Research Center (BCRC); Cat. 60153, hereinafter referred to as human skin fibrocytes.

2. Cell culture medium: a basic medium containing 10 vol % FBS (purchased from Gibco). The basic medium was prepared from an Eagle's minimal essential medium (MEM, purchased from Gibco, with the product No. 15188-319) additionally containing 1 mM sodium pyruvate (purchased from Gibco), 1.5 g/L of sodium bicarbonate (purchased from Sigma, Cat. S5761-500G) and 0.1 mM non-essential amino acid solution (purchased from Gibco).

3. Phosphate buffered saline (PBS): purchased from Gibco with the product No. 10437-028.

4. DCFH-DA solution: 2,7-dichloro-dihydro-fluorescein diacetate (DCFH-DA; product No. SI-D6883, purchased from Sigma) was dissolved in dimethyl sulfoxide (DMSO, purchased from Sigma, product No. SI-D6883-50 MG) to prepare 5 μg/mL of DCFH-DA solution.

5. Flow cytometer, BD Accuri C6 Plus.

6. Hydrogen peroxide (H₂O₂): purchased from Sigma-Aldrich, product model No. 95299-1L.

7. Trypsin (Trypsin-EDTA): 10× Trypsin-EDTA (purchased from Gibco) was diluted 10-fold in PBS.

### Test Process:

1. The human skin fibrocytes were inoculated at 1×10⁵ cells per well in a six-well culture plate containing 2 mL of the cell culture medium per well.

2. The culture plate was placed at 5% CO₂ and 37° C. for culture for 24 hours.

3. After the culture, the cells were divided into the following three groups and subjected to corresponding treatment procedures.

Blank group: the medium in each well was replaced with 2 mL of pure cell culture medium (that is, not containing the peach extract of the present invention).

Control group: the medium in each well was replaced with 2 mL of pure cell culture medium.

TCI-AP-01 group: the medium in each well was replaced with 2 mL of cell culture medium containing hyperoside (separated from Example VII) at a concentration of 0.1 mM.

TCI-AP-02 group: the medium in each well was replaced with 2 mL of cell culture medium containing isoquercitrin (separated from Example VII) at a concentration of 0.1 mM.

TCI-AP-07 group: the medium in each well was replaced with 2 mL of cell culture medium containing nicotiflorin (separated from Example VII) at a concentration of 0.1 mM.

TCI-AP-08 group: was replaced with in each well was replaced with 2 mL of cell culture medium containing rutin (separated from Example VII) at a concentration of 0.1 mM.

4. After the culture medium was replaced, the culture plate was placed at 5% CO₂ and 37° C. for culture for 1 hour.

5. 2 μL of DCFH-DA solution was added to each well for reaction for 15 minutes.

6. After the treatment with the DCFH-DA, H₂O₂ was added in the experimental groups and the control group to react at 37° C. for 1 hour. In other words, 1 mM hydrogen peroxide was added to each group to simulate oxidative damage.

7. Then the cells in each well were rinsed twice with 1 mL of PBS.

8. 200 μL of trypsin was added to each well to react at a dark place for 5 minutes. After the reaction, the cell culture medium was added to each well to terminate the reaction.

9. The cells and the cell culture medium of each group were collected into individual corresponding centrifuge tubes, and the centrifuge tubes containing the cells and the cell culture medium were centrifuged at 400×g for 10 minutes.

10. After the centrifugation, a supernatant in each centrifuge tube was removed, and a cell precipitate in each centrifuge tube was redissolved with PBS.

11. Next, each centrifuge tube was centrifuged again at 400×g for 10 minutes.

12. After the re-centrifugation, a supernatant in each centrifuge tube was removed and a cell precipitate was suspended with 1 mL of PBS at a dark place to obtain a cell fluid to be tested.

13. The fluorescent signal of DCFH-DA in the cell fluid to be tested in each tube was detected by using a flow cytometer to quantify the content of intracellular ROS. The excitation wavelength used for detecting the fluorescence signal was 450-490 nm, and the emission wavelength used therefor was 510-550 nm. Since the test was performed in triplicate, the measurement results of the triplicate test in each group were averaged to obtain an average value as the content of ROS.

### Test Results:

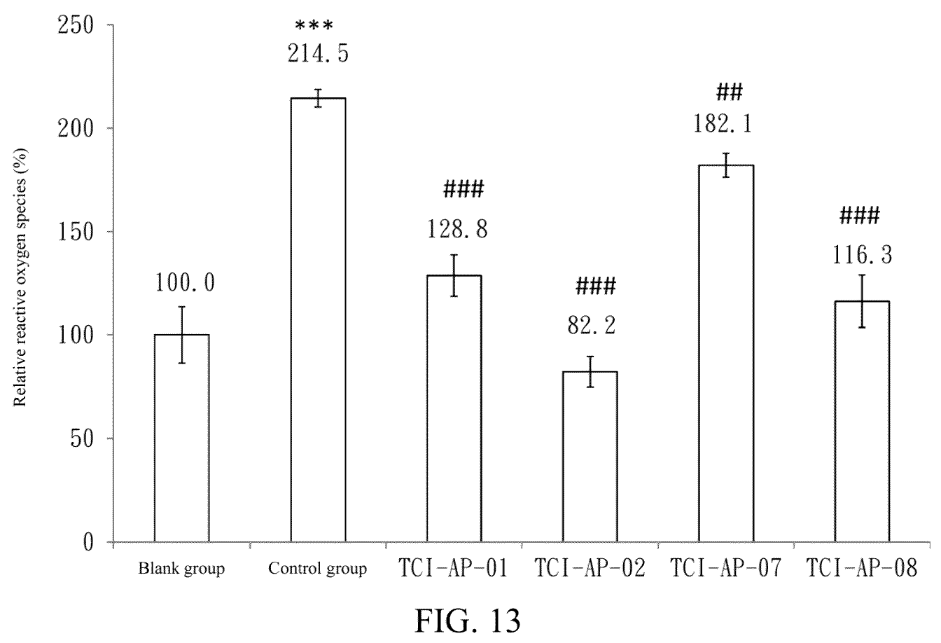

Herein, the human skin fibrocytes of the blank group were not subjected to damage by hydrogen peroxide. It also means that they were under normal physiological metabolic conditions. Therefore, it is assumed that its relative ROS production amount was 100%. Besides, when the content of ROS of the blank group was regarded as 100% of the relative ROS production amount, the content of ROS of each of the remaining groups was converted into the relative ROS production amount expressed in % by an interpolation method as shown in Table 4 and FIG. 13.

TABLE 4

| | Relative ROS production amount |
|---|---|
| Blank group | 100% |
| Control group | 214.5% |
| TCI-AP-01 | 128.8% |
| TCI-AP-02 | 82.2% |
| TCI-AP-07 | 182.1% |
| TCI-AP-08 | 116.3% |

In FIG. 13, compared with the blank group, “*” represents a p value less than 0.05, “**” represents a p value less than 0.01, and “***” represents a p value less than 0.001; and compared with the control group, “#” represents a p value less than 0.05, “##” represents a p value less than 0.01, and “###” represents a p value less than 0.001. The more “*” or “#” represents the more statistically significant difference.

As shown in FIG. 13, by comparing the results of the blank group and the control group, it can be known that after the treatment with the hydrogen peroxide, the relative ROS production amount (high fluorescence expression) was greatly increased by more than two times, statistically significant “***”, and a p value is less than 0.001; and it indicates that the hydrogen peroxide treatment did promote the production of intracellular ROS, which can damage skin fibroblasts.

Still refer to Table 4 and FIG. 13. After the human skin fibrocytes of the TCI-AP-01 group were treated with the hyperoside, even if the strong oxidative damage was simulated by the hydrogen peroxide, the relative ROS production amount also tends to decline. In other words, compared with the relative ROS production amount of the control group, the relative ROS production amount of the TCI-AP-01 group was significantly reduced to 128.8%.

After the human skin fibrocytes of the TCI-AP-02 group were treated with the isoquercitrin, even if the strong oxidative damage is simulated by the hydrogen peroxide, the relative ROS production amount also tends to decline. In other words, compared with 100% of the relative ROS production amount of the blank group, even if the strong oxidative damage was simulated by the hydrogen peroxide, the relative ROS production amount of the TCI-AP-02 group was reduced to 82.2% compared with that under normal physiological metabolism.

After the human skin fibrocytes of the TCI-AP-07 group were treated with the nicotiflorin, even if the strong oxidative damage was simulated by the hydrogen peroxide, the relative ROS production amount also tends to decline. In other words, compared with the relative ROS production amount of the control group, the relative ROS production amount of the TCI-AP-07 group was significantly reduced to 182.1%.

After the human skin fibrocytes of the TCI-AP-08 group were treated with the rutin, even if the strong oxidative damage was simulated by the hydrogen peroxide, the relative ROS production amount also tends to decline. In other words, compared with the relative ROS production amount of the control group, the relative ROS production amount of the TCI-AP-08 group was significantly reduced to 116.3%.

The above test results show that the bioactive substances of the peach extract of this example can effectively reduce the production or accumulation of ROS in the cells.

In conclusion, according to any embodiment of the present invention, the peach extract can be used in the preparation of a composition for improving skin condition. In other words, the peach extract has at least one of the following effects: resisting blue light-induced injury, improving oxidation resistance of skin fibrocytes, improving activity of mitochondria, promoting production of skin elastin, improving skin texture, reducing skin wrinkles, minimizing skin trichopores, improving the content of heme, improving the content of iron in blood, and the like.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for improving skin condition of a subject in need thereof, comprising administering to the subject an effective amount of peach extract, wherein the peach extract is extracted from red flesh of peaches.

2. The method according to claim 1, wherein the peach extract resists blue light-induced injury of the subject to achieve the effect of improving skin condition.

3. The method according to claim 1, wherein the peach extract improves activity of mitochondria of the subject to achieve the effect of improving skin condition.

4. The method according to claim 1, wherein the peach extract promotes production of skin elastin of the subject to achieve the effect of improving skin condition.

5. The method according to claim 1, wherein the peach extract improves skin texture of the subject to achieve the effect of improving skin condition.

6. The method according to claim 1, wherein the peach extract reduces skin wrinkles of the subject to achieve the effect of improving skin condition.

7. The method according to claim 1, wherein the peach extract minimize skin trichopores of the subject to achieve the effect of improving skin condition.

8. The method according to claim 1, wherein the peach extract improves oxidation resistance of skin fibrocytes of the subject to achieve the effect of improving skin condition.

9. The method according to claim 1, wherein the peach extract comprises at least hyperoside, isoquercitrin, nicotiflorin, and rutin.

10. The method according to claim 9, wherein the hyperoside, the isoquercitrin, the nicotiflorin, and the rutin resist blue light-induced injury of the subject to achieve the effect of improving skin condition.

11. The method according to claim 9, wherein the hyperoside, the isoquercitrin, the nicotiflorin, and the rutin improve oxidation resistance of skin fibrocytes of the subject to achieve the effect of improving skin condition.

12. A method for relieving anemia of a subject in need thereof, comprising administering to the subject an effective amount of peach extract, wherein the peach extract is extracted from red flesh of peaches.

13. The method according to claim 12, wherein the peach extract improves the content of heme of the subject to achieve the effect of relieving anemia.

14. The method according to claim 12, wherein the peach extract improves the content of iron in blood of the subject to achieve the effect of relieving anemia.

15. The method according to claim 12, wherein the peach extract comprises at least hyperoside, isoquercitrin, nicotiflorin, and rutin.

16. The method according to claim 15, wherein the hyperoside, the isoquercitrin, the nicotiflorin, and the rutin resist blue light-induced injury of the subject to achieve the effect of relieving anemia.

17. The method according to claim 15, wherein the hyperoside, the isoquercitrin, the nicotiflorin, and the rutin improve oxidation resistance of skin fibrocytes of the subject to achieve the effect of relieving anemia.
